(19) United States
(12) Patent Application Publication
(10) Pub. No.: US 2020/0010399 A1
(43) Pub. Date: Jan. 9, 2020

(54) METHOD OF MAKING DIALKYL TEREPHTHALATE FROM TEREPHTHALIC ACID

(71) Applicant: SABIC Global Technologies B.V., Bergen op Zoom (NL)

(72) Inventors: Ritesh Nandy, Bangalore (IN); Vishal Patrick, Bangalore (IN); Edward Joseph Nesakumar, Bangalore (IN)

(21) Appl. No.: 16/493,034

(22) PCT Filed: Mar. 15, 2018
(86) PCT No.: PCT/IB2018/051754

§ 371 Date: Sep. 11, 2019

Related U.S. Application Data
(60) Provisional application No. 62/471,623, filed on Mar. 15, 2017.

(51) Int. Cl. C07C 67/08

(52) U.S. Cl. CPC C07C 67/08; C07C 69/82

(57) ABSTRACT

A method for the manufacture of a diester of terephthalic acid includes reacting terephthalic acid and a C₆₋₁₈ alkyl monohydric alcohol in the presence of a catalyst having the formula MO[CH₃COCH═C(0-)CH₃]₂ wherein M=Ti, Zr, or Hf, under conditions effective to form a reaction mixture comprising the di(C₆₋₁₈ alkyl) terephthalate.

### BACKGROUND

This disclosure relates to a method of manufacture of a dialkyl terephthalate, such as dioctyl terephthalate, from terephthalic acid.

Plasticizers are widely used in plastics, coating compositions, sealing compositions, and rubber articles.

Dioctylphthalate (DOP) has been one of the most extensively used plasticizers in plastics processing. DOP has been used in processing polyvinyl chloride and ethylcellulose resins to produce plastic film, imitation leather, electric wire, cable wearer, sheet, planet, mold plastic products, and the like.

However, the ongoing public debate over whether use of phthalates has negative health impacts has led to a shrinking market demand, especially in the European market, for the most commonly used phthalates, including DOP. In response to the ongoing scrutiny of phthalates, producers of plasticizers have worked to develop alternative, phthalate free plasticizers, such as dioctyl terephthalate (DOTP).

Although synthetic methods to obtain DOTP from terephthalic acid (TPA) or dimethyl terephthalate (DMT) are known, the methods are slow and can cause foaming Alternative methods for synthesis of DOTP involve reaction at elevated pressure in the presence of titanium catalyst, but result in conversion of only about 80-90% of the TPA.

According, there is a need in the art for improved processes for synthesizing diesters of terephthalic acid, such as DOTP providing higher terephthalic acid conversion and higher yields of the desired isolated product.

### BRIEF DESCRIPTION

A method for the manufacture of a diester of terephthalic acid includes reacting terephthalic acid and a C₆₋₁₈ alkyl monohydric alcohol in the presence of a catalyst having the formula MO[CH3COCH═C(O—)CH₃]₂ wherein M=Ti, Zr, or Hf, under conditions effective to form a reaction mixture comprising the di(C₆₋₁₈ alkyl) terephthalate.

A method to prepare dioctyl terephthalate comprises reacting terephthalic acid and 2-ethyl hexanol at a molar ratio of 2-ethyl hexanol to terephthalic acid of 2.6:1 to 4.2:1, in the presence of 0.10 mole percent to 0.30 mol percent of a catalyst having the formula TiO[CH₃COCH═C(O—)CH₃]₂under conditions effective to form a reaction mixture comprising dioctyl terephthalate in a yield of at least 95 percent, preferably at least 97%, more preferably at least 98%, based on the terephthalic acid; and removing unreacted terephthalic acid, 2-ethyl hexanol, and catalyst from the reaction mixture, to provide an isolated dioctyl terephthalate.

A di(C₆₋₁₈ alkyl) terephthalate made by the above process is also disclosed

The above described and other features are exemplified by the following detailed description.

### DETAILED DESCRIPTION

A method for manufacture of dialkyl terephthalates from terephthalic acid (TPA) is disclosed. The method includes esterification of terephthalic acid with a C₆₋₁₈ alkyl monohydric alcohol in the presence of a metal oxyacetylacetonate catalyst. The method provides higher conversion of terephthalic acid and higher isolated yield of the dialkyl terephthalate product compared to other known processes, with excellent selectivity and high TPA conversion. The higher product yield from the disclosed method is advantageous in commercial manufacture of dialkyl terephthalate. The method is particularly useful for the production of DOTP, and proceeds in high yield.

Examples of the C₆₋₁₈ alkyl monohydric alcohol include a saturated, straight or branched-chain aliphatic alcohol such as normal- or iso-hexanol, normal- or iso-heptanol, normal- or iso-octanol, 2-ethylhexanol, normal- or iso-nonyl alcohol, normal- or iso-decanol, 2-propyl heptanol, normal- or iso-undecanol, normal or iso-dodecanol, or normal- or iso-tridecanol; or a saturated monohydric cycloaliphatic alcohol such as cyclohexanemethanol and methylcyclohexanemethanol (including cis and trans isomers, as well as the 1,2-, 1,3-, or 1,4-isomers); or the like; or a combination comprising at least one of the foregoing. Preferably C₆₋₁₃ alcohols are used, and the C₇₋₁₀ alcohols, especially the C₈ alcohols, are preferred in the production of the di(C₆₋₁₈alkyl) terephthalates.

In some embodiments, the C₆₋₁₈ alcohol can comprise n-hexanol, cyclohexanol, n-heptanol, 2-ethylhexanol, cyclohexanemethanol, n-octanol, n-nonanol, n-decanol, or a combination comprising at least one of the foregoing. For example, the C₆₋₁₈ alcohol can be 2-ethylhexanol. When the C₆₋₁₈ alcohol is 2-ethylhexanol, the di(C₆₋₁₈alkyl) terephthalate is known as DOTP in the art.

The reaction between TPA and the C₆₋₁₈ monohydric alcohol is conducted in the presence of a catalytic amount of metal oxyacetylacetonate catalyst. The metal can comprise titanium, zirconium, hafnium, or a combination comprising at least one of the foregoing. For example the catalyst can be titanium oxyacetylacetonate.

The TPA and the C₆₋₁₈ monohydric alcohol can be present in an amount effective to provide a molar ratio of TPA to alcohol of 1:2 to 1:10, for example 1:2.5 to 1:6, preferably from 1:3 to 1:4, more preferably 1:3.2 to 1: 3.6.

The catalyst can be present in an amount of 0.05 to 0.5 mole percent (mol %), or 0.1 to 0.4 mole %, or 0.2 to 0.35 mol %, or 0.15 to 0.3 mol %, based on the moles of TPA. In an embodiment, the catalyst is titanium oxyacetylacetonate present in an amount of 0.17 to 0.25 mol %, based on the total moles of TPA.

The reaction is initiated by combining the TPA, the C₆₋₁₈ alcohol, and the catalyst to obtain a reaction mixture, and then heating the reaction mixture to provide a product mixture. Preferably, the reaction mixture is homogenous. The reaction is carried out under conditions effective to provide the di(C₆₋₁₈alkyl) terephthalate, for example DOTP. A variety of conditions can be suitable for the reaction, depending on the particular alcohol, desired efficiency, catalyst, and other considerations. For example, the reaction can be carried out at a temperature of 160 to 250° C., for example 170 to 230° C., or 170 to 210° C. or 200 to 220° C., and a pressure of 0.3 to 10 bar. The reaction is preferably carried out at atmospheric pressure, unless the boiling point of the C₆₋₁₈ alcohol is less than the reaction temperature. In such cases, the reaction is carried out under pressure. Furthermore, the reaction is conducted for a suitable period of time, for example 12 to 36 hours, or for 20 to 28 hours. The reaction can be conducted under an inert atmosphere, e.g., under nitrogen or argon.

In some embodiments, the product mixture can comprise the di(C₆₋₁₈alkyl) terephthalate, specifically DOTP, residual C₆₋₁₈ alcohol, water, unreacted TPA, one or more by-products, or a combination comprising at least one of the foregoing. The reaction can be conducted with the concomitant removal of one or more of these, for example water, a by-product, or a combination thereof. Alternatively, water or the by-product can he removed separately, or after the esterification is completed.

The method further comprises isolating the di(C₆₋₁₈alkyl) terephthalate, in particular DOTP, from the product mixture. Isolating the di(C₆₋₁₈alkyl) terephthalate can include a series of process steps including one or more of distillation, acid neutralization, and filtration, which can be conducted in any order. In an embodiment, the product mixture is distilled to remove at least a portion of the residual C₆₋₁₈ alcohol, and optionally water, a by-product, or a combination thereof. Distillation can be conducted so as to remove these components sequentially or at the same time. In an embodiment, the distillation is conducted to as to provide the C₆₋₁₈ alcohol in high purity, e.g., the distilled C₆₋₁₈ alcohol has a purity of greater than 98%, or greater than 99%, as determined by gas chromatography.

The acid catalyst in the product mixture can be neutralized. In some embodiments, distilling the reaction mixture can be carried out before neutralizing the acid catalyst. In other embodiments, neutralizing the acid catalyst can be carried out before distilling the reaction mixture. In either embodiment, neutralizing the catalyst can first comprise cooling the product mixture to a temperature of less than 100° C., then adding an aqueous alkaline solution. The amount of aqueous alkaline solution that is added is generally equivalent to the amount of acid present in the reaction mixture. Exemplary bases suitable for use in the aqueous alkaline solution include alkali metal salts, particularly sodium salts such as sodium carbonate, and alkali metal hydroxides such as sodium hydroxide, e.g., aqueous sodium hydroxide.

The distillation and neutralization can produce a first intermediate mixture. In some embodiments, the first intermediate mixture can be further distilled to remove water and a final portion of the residual C₆₋₁₈ alcohol to provide a second intermediate mixture.

The first or the second intermediate mixture can be filtered to provide a filtrate comprising the di(C₆₋₁₈allcyl) terephthalate. In some embodiments, isolating the di(C₆₋₁₈₁alkyl) terephthalate further comprises treating the filtrate with a decolorizing agent such as activated charcoal, and filtering the treated filtrate, for example using a filter aid, to provide the isolated dialkyl arylate, in particular an isolated DOTP.

The method described herein can provide the di(C₆₋₁₈alkyl) terephthalate, in particular DOTP, with a selectivity of greater than 95%, for example, greater than 98%, for example, greater than 99%. A selectivity of greater than 95% means that the product comprises the di(C₆₋₁₈alkyl) terephthalate, in particular DOTP, and less than 5 weight percent (wt %) of the isophthalate diester based on the weight of the product. Similarly, a selectivity of greater than 98% means that the product comprises the di(C₆₋₁₈alkyl) terephthalate, in particular DOTP, and less than 2 wt % of the isophthalate diester, based on the weight of the product, and a selectivity of greater than 99% means that the product comprises the di(C₆₋₁₈alkyl) terephthalate, in particular DOTP, and less than 1 wt % of the isophthalate ester, based on the weight of the product.

In some embodiments, the conversion of the TPA to the di(C₆₋₁₈alkyl) terephthalate, in particular DOTP, can be greater than 85%, for example, greater than 90%, for example, greater than 95%, for example, greater than 98%, for example, greater than 99%, based on the moles of TPA. The product can comprise less than 5 wt %, preferably less than 2 wt %, more preferably less than 1 wt % of the corresponding isophthalate diester, based on the weight of the product.

The di(C₆₋₁₈alkyl) terephthalate, in particular the DOTP prepared according to the above-described method can have an APHA color of less than or equal to 25, as determined according to ASTM D1209, or less than or equal to 24.

In a specific embodiment, a method for the preparation of DOTP comprises combining TPA, a molar excess of 2-ethylhexanol, and a catalyst comprising titanium oxyacetylacetonate to provide a reaction mixture; and heating the reaction mixture at a temperature greater than 130° C., under conditions effective to provide a product mixture comprising the di(2-ethylhexyl) terephthalate. The ratio of moles of the TPA to moles of the 2-ethylhexanol can be 1:2.2 to 1:30, preferably 1:3 to 1:20, and the catalyst can be present in an amount of 0.1 to 1 mol %, based on the moles of the TPA. Heating the reaction mixture can be at a temperature of 160 to 260° C., at a pressure of 0.2 to 20 bar, for 12 to 36 hours, preferably 18 to 30 hours. The method can further comprise removing water, or other by-products.

The product mixture comprising DOTP can further comprise residual 2-ethylhexanol, water, one or more by-products, unreacted TPA, or a combination comprising at least one of the foregoing. Isolating the DOTP can be performed by distilling the product mixture to remove at least a portion of the residual 2-ethylhexanol from the product mixture; and neutralizing the product mixture. In an embodiment, distillation is conducted to provide 2-ethylhexanol having a purity of greater than 98%, or greater than 99%, as determined by GC. Neutralizing the product mixture can comprise cooling the product mixture to a temperature less than 100° C., then adding an aqueous base. Isolation can further comprise removing any solids from the product mixture, preferably by filtering the product mixture to provide a filtrate comprising the DOTP, preferably wherein removing any solids comprises filtering the distilled and neutralized product mixture to provide a filtrate comprising the DOTP. Optionally the product mixture can be treated with activated charcoal. Preferably the distilled and neutralized product mixture is treated, and more preferably the filtrate is treated with activated charcoal. The DOTP product can comprise less than 5 wt %, preferably less than 2 wt %, more preferably less than 1 wt % of the isophthalate diester, based on the weight of the product. The DOTP product can comprise less than 5 wt %, preferably less than 2 wt %, more preferably less than 1 wt % of the corresponding mono(2-ethyl hexyl) terephthalate isophthalate ester, based on the weight of the product. The yield of the DOTP can be greater than 85%, preferably greater than 90%, more preferably greater than 95%, even more preferably greater than 98%, even more preferably greater than 99%, based on the moles of TPA. The DOTP can advantageously have an APHA color of less than 25, determined according to ASTM D1209.

Further described herein are polymer compositions comprising a polymer and the di(C₆₋₁₈alkyl) terephthalate, in particular the di(2-ethylhexyl) terephthalate manufactured as described above. The di(C₆₋₁₈alkyl) terephthalate can be used as a plasticizer in a variety of polymers, particularly PVC, or cellulose acetate-butyrate, cellulose nitrate, polymethyl methacrylate, polystyrene, or polyvinyl butyral. The polymer compositions can be used to manufacture a wide variety of articles, for example beverage closures, sealing materials used in construction joints, and components of medical devices.

The methods of the present disclosure are further illustrated by the following examples, which are non-limiting.

### EXAMPLES

### Materials.

The materials used are shown in Table 1.

TABLE 1

| Component | Source |
|---|---|
| Terephthalic acid (TPA) | ≥98%, Sigma-Aldrich |
| 2-Ethylhexanol (2-EH) | 99%, SABIC |
| Titanium tetraisopropoxide (Ti-1) | ≥97%, Sigma-Aldrich |
| Titanium oxyacetylacetonate (Ti-2) | 90%, Sigma-Aldrich |
| CELITE 545 (Diatomaceous Earth) | Sigma-Aldrich |
| Charcoal, acid-washed | methylene blue - 18 ml/0.1 gm, |
| | PH - 6-7.5, SD |
| | Fine Chemical Limited |

### Analytical Methods.

APHA color was evaluated by ASTM D1209.

The acid value was determined by a titrimetric method.

High performance liquid chromatography (HPLC) was performed on a Zorbax RRHD Eclipse plus C18 3.0×100 mm with a diode array detector detecting at 254 nm wavelength.

### Example 1.

### Esterification of Terephthalic Acid (TPA) with 2-ethylhexanol (2-EH)

Esterification of TPA with 2-EH is in accordance with Scheme 1.

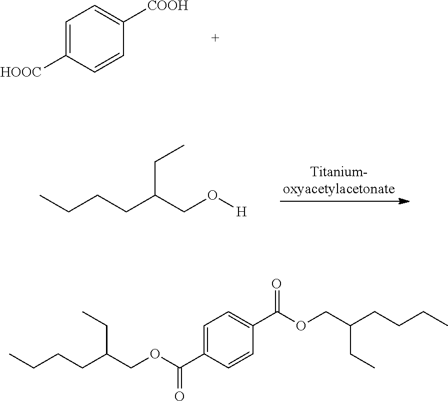

The following general procedure was used. To a four-neck round bottom flask equipped with a mechanical/magnetic stirrer, Dean-Stark apparatus, and a thermometer pocket for monitoring internal temperature, 100 g TPA and 3.5 equivalents (eq) of 2-EH were added with agitation. The mixture was heated over an oil bath with a set temperature of 210° C. The titanium catalyst was added when the internal temperature was 170° C. The quantity of catalyst varied from 0.11 mol % to 0.22 mol % with respect to TPA. After catalyst addition, slow formation of water was observed. The water formed during the reaction was collected in the Dean-Stark apparatus and drained out from time to time from the layer-separated mixture of water and 2-EH. The reaction was continued for 24 hours and then filtered. Conversion of TPA was determined by filtering the solids (unreacted TPA). HPLC was used to determine selectivity of the reaction.

A comparative study using titanium tetraisopropoxide (Ti-1) versus titanium oxyacetylacetonate (Ti-2) was carried out using 0.22 mol % of catalyst using the conditions described above. When the reaction was carried out in the presence of Ti-1 the reaction provided a TPA conversion of 90% at 24 hours, a selectivity to formation of DOTP of 98.4% (Table 2), and 16 to 18 ml water was collected. In contrast, reaction in the presence of Ti-2 under the same conditions resulted in 98% conversion of TPA at 24 hours, a selectivity to DOTP of 99.5%, and 20 to 21 ml water was collected.

### Example 2.

### Isolation of Dioctyl Terephthalate (DOTP)

The filtrate of Example 1 was cooled to below 100° C. and set for a distillation to remove excess 2-EH. After setting the vacuum at 7 to 8 mbar, the temperature of the reaction mixture was slowly raised in the following sequence: 130 to 150 to 200 to 210° C., to remove most of the 2-EH. The mixture cooled back to 90° C. and 1 ml of aqueous caustic (NaOH) solution (49%, w/v) and 2 ml water were added to the mixture and stirred for 30 to 40 min Excess caustic was neutralized by purging in-situ generated CO₂ in the reaction mixture for 20 to 30 min. Then the mixture was again distilled to remove water and the rest of the 2-EH following the previous distillation method.

The mixture was cooled to 120° C. and filtered over 1 g CELITE (1 wt % on the basis of the weight TPA used in the reaction) to remove the white solid (residual sodium salt of TPA and carbonate salt and Titanium salt) resulting in a viscous liquid. The liquid was treated at 60 to 65° C. for 30 to 40 min with 1 g of acid-washed charcoal to remove colored impurities, followed by filtration over 1 g CELITE to obtain the final product, DOTP, which is colorless, viscous liquid.

The results of the comparative study after esterification and isolation are shown in Table 2.

TABLE 2

| | | |
|---|---|---|
| Acid catalyst | Ti-1 | Ti-2 |

| | | | | |
|---|---|---|---|---|
| Catalyst loading | 0.22 | mol % | 0.22 | mol % |
| Reaction time | 24 | h | 24 | h |

| | | |
|---|---|---|
| Conversion of TPA | 90% | 98% |
| Selectivity to DOTP | 98.40% | 99.50% |
| Isolated yield | 79% | 86% |
| Isolated DOTP purity | 99.2% | 99.6% |
| Acid value | 0.019 | 0.017 |
| APHA | 27 | 23 |

Table 2 shows that using the Ti-2 catalyst results in improved conversion of the starting reagent TPA and higher selectivity for production of the product DOTP compared to using the TI-1 catalyst. Table 1 also shows that using the Ti-2 catalyst results in higher isolated yield (86%) and higher purity (99.6%) compared to the TI-1 catalyst, which had an isolated yield of 79% and purity of 99.2% of DOTP.

The methods described herein are further illustrated by the following aspects, which are non-limiting.

Aspect 1. A method for the manufacture of a diester of terephthalic acid includes reacting terephthalic acid and a C₆₋₁₈ alkyl monohydric alcohol in the presence of a catalyst having the formula MO[CH₃COCH═C(O—)CH₃]₂ wherein M=Ti, Zr, or Hf, under conditions effective to form a reaction mixture comprising the di(C₆₋₁₈ alkyl) terephthalate.

Aspect 2. The method of aspect 1, wherein M is Ti and the C_{6-C18} alkyl monohydric alcohol is a C₆-C₁₀alkyl monohydric alcohol, preferably a C₈ alcohol, preferably 2-ethylhexanol.

Aspect 3. The method of aspect 1 or 2, wherein the reacting is performed at atmospheric pressure, at a temperature of 160 to 250° C. or 170 to 210° C.

Aspect 4. The method of any one or more of aspects 1 to 3, wherein the molar ratio of terephthalic acid to C₆₋₁₈ monohydric alcohol is 1:2 to 1:10, or 1:2.5 to 1:6, or 1:3 to 1:4,or 1:3.2 to 1: 3.6.

Aspect 5. The method of any one or more of aspects 1 to 4, wherein the catalyst is present in an amount of 0.10 to 0.40 mole percent, or 0.2 to 0.35 mole percent, or 0.15 to 0.30 mole percent, or 0.17 to 0.25 mole percent, based on the total moles of TPA.

Aspect 6. The method of any one or more of aspects 1 to 5, further comprising removing water from the reaction mixture during the reacting.

Aspect 7. The method of any one or more of aspects 1 to 6, further comprising one or more of: removing unreacted terephthalic acid from the reaction mixture, removing unreacted C₆₋₁₈ monohydric alcohol from the reaction mixture, removing the catalyst from the reaction mixture, to provide an isolated di(C₆₋₁₈ alkyl) terephthalate in a yield of at least 95 percent, based on the terephthalic acid.

Aspect 8. The method of aspect 7, further comprising: neutralizing the product mixture; removing any solids from the product mixture; or treating the product mixture with a decolorizing agent, preferably an activated charcoal.

Aspect 9. A di(C₆₋₁₈ alkyl) terephthalate made by the method of any one or more of aspects 1 to 8.

Aspect 10. The di(C₆₋₁₈ alkyl) terephthalate of aspect 9 having a purity of at least 98 percent, preferably at least 99 percent, more preferably at least 99.5 percent, and an APHA color of less than or equal to 25.

Aspect 11. A method to prepare dioctyl terephthalate, the method comprising reacting terephthalic acid and 2-ethyl hexanol at a molar ratio of 2-ethyl hexanol to terephthalic acid of 2.6:1 to 4.2:1, in the presence of 0.10 mole percent to 0.30 mol percent of a catalyst having the formula TiO[CH₃COCH═C(O—)CH₃]₂under conditions effective to form a reaction mixture comprising dioctyl terephthalate in a yield of at least 95 percent, preferably at least 97%, more preferably at least 98%, based on the terephthalic acid; and removing unreacted terephthalic acid, 2-ethyl hexanol , and catalyst from the reaction mixture, to provide an isolated dioctyl terephthalate.

Aspect 12. The method of aspect 11, further comprising: neutralizing the product mixture; removing any solids from the product mixture; or reacting the product mixture with a decolorizing agent, preferably an activated charcoal.

Aspect 13. The method of aspect 11 or 12, wherein the reacting is performed at atmospheric pressure, and at a temperature of 160 to 250° C., preferably 170 to 210° C.

Aspect 14. The method of any one or more of aspects 11 to 13, wherein the molar ratio of 2-ethyl hexanol:terephthalic acid is 3.2:1 to 3.6:1; and the catalyst is present in an amount of 0.15 to 0.35 mole percent, more preferably 0.20 to 0.30 mole percent.

Aspect 15. The method of any one or more of aspects 11 to 14, further comprising removing water from the reaction mixture during the reacting.

Aspect 16. A dioctyl terephthalate made by the method of any one or more of aspects 11 to 15.

Aspect 17. The dioctyl terephthalate of aspect 16, having a purity of at least 98 percent, preferably at least 99 percent, more preferably at least 99.5 percent, and an APHA of less than 25.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments,” “an embodiment,” and so forth, means that a particular element described in connection with the embodiment is included in at least one embodiment described herein, and may or may not be present in other embodiments. In addition, it is to be understood that the described elements may be combined in any suitable manner in the various embodiments.

Unless specified to the contrary herein, all test standards, such as ASTM D1209, are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

The term “alkyl” means a branched or straight chain, unsaturated aliphatic hydrocarbon group, e.g., methyl, ethyl, n-propyl, i-propyl, n-butyl, s-butyl, t-butyl, n-pentyl, s-pentyl, and n- and s-hexyl. The number of carbon atoms indicated in a group is exclusive of any substituents. For example —CH₂CH₂CN is a C₂ alkyl group substituted with a nitrile. Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g., benzyl), C₇₋₁₂ alkylarylene (e.g, toluyl), C₄₋₁₂ heterocycloalkyl, C₃₋₁₂ heteroaryl, C₁₋₆ alkyl sulfonyl (—S(═O)₂- alkyl), C₆₋₁₂ arylsulfonyl (—S(═O)₂-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for the manufacture of a diester of terephthalic acid, the method comprising:
reacting terephthalic acid and a C₆₋₁₈ alkyl monohydric alcohol in the presence of a catalyst having the formula
MO[CH₃COCH═C(O—)CH₃]₂,
wherein M=Ti, Zr, or Hf, under conditions effective to form a reaction mixture comprising di(C₆₋₁₈ alkyl) terephthalate.

2. The method of claim 1, wherein M is Ti and the C₆-C₁₈ alkyl monohydric alcohol is a C₆-C₁₀ alkyl monohydric alcohol.

3. The method of claim 1, wherein the reacting is performed at atmospheric pressure, and at a temperature of 160° C. to 250° C.

4. The method of claim 1, wherein the molar ratio of terephthalic acid to C₆₋₁₈ alkyl monohydric alcohol is 1:2 to 1:10.

5. The method of claim 1, wherein the catalyst is present in an amount of 0.10 to 0.40 mole percent based on the total moles of terephthalic acid.

6. The method of claim 1, further comprising at least one of:
(i) removing water from the reaction mixture during the reacting,
(ii) removing unreacted terephthalic acid from the reaction mixture,
(iii) removing unreacted C₆₋₁₈ alkyl monohydric alcohol from the reaction mixture, or
(iv) removing the catalyst from the reaction mixture, to provide an isolated di(C₆₋₁₈ alkyl) terephthalate in a yield of at least 95 percent, based on the terephthalic acid.

7. The method of claim 6, further comprising:
neutralizing the reaction mixture;
removing solids from the reaction mixture; or
treating the reaction mixture with a decolorizing agent.

8. A di(C₆₋₁₈ alkyl) terephthalate made by the method of claims 1.

9. The di(C₆₋₁₈ alkyl) terephthalate of claim 8 having a purity of at least 98 percent, and an APHA color of less than or equal to 25.

10. A method to prepare dioctyl terephthalate, the method comprising:
reacting terephthalic acid and 2-ethyl hexanol at a molar ratio of 2-ethyl hexanol to terephthalic acid of 2.6:1 to 4.2:1, in the presence of 0.10 mole percent to 0.30 mol percent of a catalyst having the formula
TiO[CH₃COCH═C(O—)CH₃]₂,
under conditions effective to form a reaction mixture comprising dioctyl terephthalate in a yield of at least 95 percent based on the terephthalic acid; and
removing unreacted terephthalic acid, 2-ethyl hexanol, and catalyst from the reaction mixture, to provide an isolated dioctyl terephthalate.

11. The method of claim 10, further comprising:
neutralizing the reaction mixture;
removing solids from the reaction mixture; or
treating the reaction mixture with a decolorizing agent.

12. The method of claim 10, wherein the reacting is performed at atmospheric pressure, and at a temperature of 160° C. to 250° C.

13. The method of claim 10, wherein
the molar ratio of 2-ethyl hexanol to terephthalic acid is 3.2:1 to 3.6:1; and
wherein the catalyst is present in an amount of 0.15 to 0.25 mole percent.

14. The method of claim 10, further comprising removing water from the reaction mixture during the reacting.

15. A dioctyl terephthalate made by the method of claim 10.

16. The method of claim 1, wherein M is Ti and the C₆₋₁₈ alkyl monohydric alcohol is a C₈ alcohol;
wherein the molar ratio of terephthalic acid to C₆₋₁₈ alkyl monohydric alcohol is 1:3.2 to 1:3.6; and
wherein the catalyst is present in an amount of 0.17 to 0.25 mole percent, based on the total moles of terephthalic acid.

17. The method of claim 1, wherein M is Ti and the C₆₋₁₈ alkyl monohydric alcohol is 2-ethyl hexanol.

18. The method of claim 7, wherein treating the reaction mixture with a decolorizing agent includes treating the reaction mixture with an activated charcoal.

19. The di(C₆₋₁₈ alkyl) terephthalate of claim 8 having a purity of at least 99.5 percent, and an APHA color of less than or equal to 25.

20. The method of claim 2,
wherein the reacting is performed at atmospheric pressure, and at a temperature of 160° C. to 250° C.;
wherein the molar ratio of terephthalic acid to C₆₋₁₈ alkyl monohydric alcohol is 1:2 to 1:10;
wherein the catalyst is present in an amount of 0.10 to 0.40 mole percent, based on the total moles of terephthalic acid; and
further comprising at least one of:
(i) removing water from the reaction mixture during the reacting,
(ii) removing unreacted terephthalic acid from the reaction mixture,
(iii) removing unreacted C₆₋₁₈ alkyl monohydric alcohol from the reaction mixture, or
(iv) removing the catalyst from the reaction mixture, to provide an isolated di(C₆₋₁₈ alkyl) terephthalate in a yield of at least 95 percent, based on the terephthalic acid.
